(19) United States
(12) Patent Application Publication
(10) Pub. No.: US 2026/0196334 A1
(43) Pub. Date: Jul. 9, 2026

(54) INTELLIGENT RECOMMENDATION SYSTEM FOR CHINESE MEDICINE MERIDIAN PHYSIOTHERAPY SCHEME TARGETING CHRONIC PAIN

(71) Applicant: Guangdong University of Technology, Guangzhou (CN)

(72) Inventors: Kan Xie, Guangzhou (CN); Shengli Xie, Guangzhou (CN); Rongjian Qiu, Guangzhou (CN); Junjie Yang, Guangzhou (CN); Xingfan Zhang, Guangzhou (CN); Wenyao Liang, Guangzhou (CN); Qiyu Yang, Guangzhou (CN); Bo Zhang, Guangzhou (CN)

(21) Appl. No.: 19/441,692

(22) Filed: Jan. 6, 2026

(30) Foreign Application Priority Data
Jan. 7, 2025 (CN) 202510024121.8

(51) Int. Cl. G16H 20/90; G16H 10/60

(52) U.S. Cl. CPC G16H 20/90; G16H 10/60

(57) ABSTRACT

An intelligent recommendation system for a Chinese medicine meridian physiotherapy scheme targeting chronic pain is provided in the disclosure, including a multi-modal data acquisition module, a knowledge map construction module, an intelligent analysis module, and a physiotherapy scheme recommendation module. The multi-modal data acquisition module is configured to collect and preprocess multi-modal data from a patient. The knowledge map construction module is configured to construct an acupoint-finding knowledge map and a human body linkage relationship graph on the basis of Chinese medicine meridian physiotherapy experience data. The intelligent analysis module is configured to generate a set of preliminary associated acupoints. The physiotherapy scheme recommendation module is configured to generate a final personalized Chinese medicine meridian physiotherapy scheme. The disclosure enables intelligent recommendation of high-precision and personalized Chinese medicine meridian physiotherapy schemes. Meanwhile, the disclosure requires no data acquisition and features simple operation, improving diagnostic efficiency and reducing physicians' workload.

### CROSS-REFERENCE TO RELATED APPLICATION

This application claims priority of Chinese Patent Application No. 202510024121.8, filed on Jan. 7, 2025, the entire contents of which are incorporated herein by reference.

### TECHNICAL FIELD

The disclosure relates to the technical fields of deep learning and Chinese medicine-assisted diagnostic and therapeutic, and more specifically, relates to an intelligent recommendation system for a Chinese medicine meridian physiotherapy scheme targeting chronic pain.

### BACKGROUND

According to the “China Pain Medical Development Report (2020)”, the number of patients with chronic pain in China has reached as high as 300 million and is growing at a rate of 10 million to 20 million per year. Chronic pain can be alleviated by Chinese medicine physiotherapy methods such as acupuncture, moxibustion, or electrical stimulation, and the precise recommendation of high-precision and personalized Chinese medicine meridian physiotherapy schemes helps to improve the therapeutic efficacy of Chinese medicine meridian physiotherapy. In the field of chronic pain management, the existing Chinese medicine diagnostic methods represented by acupuncture and Tuina mainly rely on the experience and skills of physicians. Taking the existing skin acupuncture therapy, a pain treatment method based on Chinese medicine theory, as an example, it determines the pain type and intensity of patients through comprehensive symptom inquiry and myofascial trigger point localization. This Chinese medicine physiotherapy scheme is characterized by quickly identifying a pain location through meticulous inquiry and palpation by physicians and providing a personalized physiotherapy scheme on the basis of Chinese medicine expert's summarized acupuncture experience. However, the current myofascial trigger point localization method mainly depends on the inquiry and palpation by physicians, highly demanding on the experience and skills of physicians, and time-consuming, thereby making it difficult to meet the growing needs of a large number of pain patients. Additionally, both the variations in pain sensitivity and subjective description errors of patients during palpation can affect diagnostic accuracy.

With the advancement of science and technology, various rehabilitation physiotherapy products based on digital health monitoring devices are commercially available. For example, some products integrate wearable devices worn at fixed positions on the human body, enabling real-time monitoring of physiological parameters of patients and providing pain management advice through data analysis. The function of these technologies is to assist physicians in diagnostic and therapeutic through objective physiological data.

Existing chronic pain management technologies, whether Chinese medical methods or modern technological methods, all have certain limitations. The former overly relies on the personal experience and techniques of physicians, and the subjective statements of patients, and results in low efficiency in implementing consultation tailored to individual differences. The latter, although providing objective data, requires regular wearing device at fixed positions, which affects daily life, and the data collection and analysis process often lacks personalized and in-depth symptom analysis. Therefore, it is urgent to develop an intelligent inquiry and recommendation system by combining patient condition information with a knowledge base of physicians, to improve inquiry efficiency and accuracy.

### SUMMARY

To overcome the shortcomings of traditional methods in the prior art, including excessive reliance on physicians' experience, strong subjectivity, low inquiry efficiency, and regular wearing of digital device, and lacking personalized and in-depth symptom analysis, the disclosure provides an intelligent recommendation system for a Chinese medicine meridian physiotherapy scheme targeting chronic pain. Diagnostic efficiency is improved and the workload of physicians is reduced while personalized medical inquiries are achieved. Moreover, there is no need to wear a fixed device for data collection. The system is simple to operate, easy to generalize, and allows non-professionals to conduct self-directed pain inquiries and rehabilitation management.

To solve the above-mentioned technical problems, the disclosure provides the following technical solutions.

An intelligent recommendation system for a Chinese medicine meridian physiotherapy scheme targeting chronic pain includes a multi-modal data acquisition module, a knowledge map construction module, an intelligent analysis module, and a physiotherapy scheme recommendation module.

The multi-modal data acquisition module is sequentially connected to the intelligent analysis module and the physiotherapy scheme recommendation module; and the knowledge map construction module is connected to the physiotherapy scheme recommendation module.

The multi-modal data acquisition module is configured to collect and preprocess multi-modal data from a patient with chronic pain, the multi-modal data at least including medical history information, a pain location, a pain type, and personalized movement examination feedback data.

The knowledge map construction module is configured to construct an acupoint-finding knowledge map and a human body linkage relationship graph on the basis of preset Chinese medicine meridian physiotherapy experience data.

The intelligent analysis module is internally preset with a multi-modal attention network, which is configured to perform feature extraction, feature fusion, and preliminary prediction of acupoints on preprocessed multi-modal data, and generate a set of preliminary associated acupoints.

The physiotherapy scheme recommendation module is configured to obtain a general physiotherapy scheme on the basis of the acupoint-finding knowledge map, input the general physiotherapy scheme, the set of preliminary associated acupoints, and the human body linkage relationship graph into a preset optimization and calibration network, and ultimately generate a personalized Chinese medicine meridian physiotherapy scheme and complete intelligent recommendation.

In an embodiment, the multi-modal data is acquired based on the patient's chief complaints, with medical history information including at least pain area, pain type, pain intensity, pain duration, past history of pain, treatment records, and medication usage;
the pain location includes at least one of head, neck, scapular region, chest, back, waist, pelvis and hip region, legs, knees, ankles, feet, elbows, wrists, or fingers;
the pain type includes dull pain or sharp pain, where the dull pain at least includes aching pain, distending pain, and stuffy pain, and the sharp pain at least includes bursting pain, cutting pain, pricking pain, burning pain, and colicky pain; and
on the basis of the pain area selected by the patient, the patient completes movement examinations in horizontal plane, coronal plane, and sagittal plane according to a preset movement prompt, each movement is scored by a visual analog scale (VAS), and a VAS score is used as the personalized movement examination feedback data of the patient.

In an embodiment, the preprocessing includes: performing information coding on the multi-modal data, the information coding including mapping and standardizing the multi-modal data according to a predefined data model; and
performing duplicate data removal, missing value filling, format error correction, and outlier processing on the multi-modal data after information coding to complete the preprocessing.

In an embodiment, in the knowledge map construction module, the preset Chinese medicine meridian physiotherapy experience data is imported into a graph database to form the acupoint-finding knowledge map; and
in the knowledge map construction module, on the basis of the preset Chinese medicine meridian physiotherapy experience data, an adjacency matrix graph including mutual strength relationships among at least 90 diagnostic and therapeutic points is constructed according to strength characteristics associated with human body linkages, and the human body linkage relationship graph is obtained; and in the human body linkage relationship graph, nodes represent different acupoints, and edges represent direct associations among acupoints.

In an embodiment, the preset Chinese medicine meridian physiotherapy experience data specifically is medical case data related to Chinese medicine meridian physiotherapy obtained from a plurality of sources.

In an embodiment, in the intelligent analysis module, a structure of the multi-modal attention network includes an embedding layer, a multi-head self-attention layer, a mapping layer, a mutual attention layer, and a first multi-layer perceptron layer which are sequentially connected;
the embedding layer being configured to perform key feature extraction on inputted preprocessed multi-modal data, and map a discrete variable in the preprocessed multi-modal data to a continuous vector space to obtain a multi-modal embedding vector;
the multi-head self-attention layer being configured to perform adaptive feature weighting of the multi-modal embedding vector;
the mapping layer being configured to project the multi-modal embedding vector after passing through the multi-head self-attention layer into a higher-dimensional vector space to obtain a high-dimensional multi-modal vector;
the mutual attention layer, on the basis of the mutual attention mechanism, being configured to perform feature fusion on the high-dimensional multi-modal vector, capture mutual dependencies among different modalities by calculating similarity weights among the different modalities, and generate a fused feature after weighting the mutual dependencies; and
the first multi-layer perceptron layer being configured to perform preliminary probability prediction of the acupoints on the basis of the fused feature, and output the set of preliminary associated acupoints formed by top-ranked acupoints in terms of prediction probability.

In an embodiment, in the physiotherapy scheme recommendation module, the optimization and calibration network includes a graph attention layer and a second multi-layer perceptron layer which are sequentially connected;
the general physiotherapy scheme, the set of preliminary associated acupoints, and the human body linkage relationship graph are jointly inputted into the graph attention layer, and the graph attention layer is configured to perform relationship modeling and semantic enhancement on the general physiotherapy scheme and the set of preliminary associated acupoints by using the human body linkage relationship graph, and calculate a deep nonlinear feature of a physiotherapy scheme containing human body linkage relationships; and
the second multi-layer perceptron layer is configured to optimize and calibrate the deep nonlinear feature, capture an implicit combination pattern of the acupoints, output a final acupoint prediction result, and save the final acupoint prediction result as the personalized Chinese medicine meridian physiotherapy scheme, to complete the intelligent recommendation.

In an embodiment, the system further includes a feedback and update module, where the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

In an embodiment, the patient with chronic pain receives treatment according to the personalized Chinese medicine meridian physiotherapy scheme, and feeds back efficacy data to the feedback and update module; and
a preset loss function is calculated on the basis of the efficacy data, and on the basis of the preset loss function, the acupoint-finding knowledge map is updated, and the multi-modal attention network and the optimization and calibration network are fine-tuned.

In an embodiment, the preset loss function is specifically:

$$Loss = min Δ RI ( p )$$

where Loss is a value of the preset loss function; and ΔRI(p) is a rehabilitation index obtained on the basis of the efficacy data.

Compared with the prior art, the disclosure has the following advantageous effects.

An intelligent recommendation system for a Chinese medicine meridian physiotherapy scheme targeting chronic pain provided in the disclosure includes a multi-modal data acquisition module, a knowledge map construction module, an intelligent analysis module, and a physiotherapy scheme recommendation module. The multi-modal data acquisition module is configured to collect and preprocess multi-modal data from a patient with chronic pain. The knowledge map construction module is configured to construct an acupoint-finding knowledge map and a human body linkage relationship graph on the basis of preset Chinese medicine meridian physiotherapy experience data. The intelligent analysis module is internally preset with a multi-modal attention network, and the multi-modal attention network is configured to perform feature extraction, feature fusion, and preliminary prediction of the acupoints on preprocessed multi-modal data, and generate a set of preliminary associated acupoints. The physiotherapy scheme recommendation module is internally preset with an optimization and calibration network, which is configured to generate a personalized Chinese medicine meridian physiotherapy scheme by using a general physiotherapy scheme provided by an acupoint-finding knowledge map, the set of preliminary associated acupoints calculated by a model, and the human body linkage relationship graph, thereby completing intelligent recommendation.

In the disclosure, by integrating the multi-modal data and an attention-based neural network, a pain location and type can be more accurately identified, and a personalized Chinese medicine meridian physiotherapy scheme is provided. The recommendation accuracy of this system can rival the diagnosis of professional doctors, making treatments more targeted. Moreover, through automated inquiry and movement examination processes, the system reduces the communication time between doctors and patients during traditional inquiries, alleviating the workload of doctors. Patients can also receive treatment recommendations more quickly, minimizing waiting times, thereby effectively improving inquiry efficiency. Additionally, on the basis of the constructed knowledge map, the system can simulate inquiry experiences of doctors and offer professional treatment suggestions, without requiring the wearing of fixed devices for data collection. The system is simple in operation and easy in promotion, meaning that, in certain situations, ordinary individuals can use this system for self-treatment or auxiliary treatment, reducing the reliance on professional doctors.

### BRIEF DESCRIPTION OF THE DRAWINGS

FIG. 1 is a structural diagram of an intelligent recommendation system for a Chinese medicine meridian physiotherapy scheme targeting chronic pain according to Embodiment 1.

FIG. 2 is a structural diagram of an intelligent recommendation system for a Chinese medicine meridian physiotherapy scheme targeting chronic pain according to Embodiment 2.

FIG. 3 is an operation flowchart of an intelligent recommendation system for a Chinese medicine meridian physiotherapy scheme targeting chronic pain according to Embodiment 2.

FIG. 4 is a schematic diagram showing model training of a system according to Embodiment 2.

FIG. 5 is a schematic diagram showing human body movement examination according to Embodiment 2.

FIG. 6 is a schematic diagram of a multi-head self-attention layer according to Embodiment 2.

FIG. 7 is a schematic diagram showing the alignment of patient chief complaint data according to Embodiment 2.

FIG. 8 is a schematic diagram of an acupoint-finding knowledge map according to Embodiment 2.

FIG. 9 is a schematic diagram of a human body linkage relationship according to Embodiment 2.

FIG. 10 is a schematic diagram showing a structure of an optimization and calibration network and inference process according to Embodiment 2.

FIG. 11 is a schematic diagram of a feedback and update knowledge map and model parameter fine-tuning according to Embodiment 2.

FIG. 12A is a diagram showing the statistics (the number of patients) of gender when doctor diagnosis is applied according to Embodiment 3;

FIG. 12B is a diagram showing the statistics (the number of patients) of gender when device diagnosis is applied according to Embodiment 3;

FIG. 12C is a diagram showing the statistics (the number of patients) of age group when doctor diagnosis is applied according to Embodiment 3; and

FIG. 12D is a diagram showing the statistics (the number of patients) of age group when device diagnosis is applied according to Embodiment 3.

FIG. 13 is a comparison diagram showing the statistics (number of patients) of pain locations between an experimental group and a control group according to Embodiment 3.

FIG. 14 is a schematic diagram of an intelligent Chinese medicine meridian physiotherapy instrument according to Embodiment 3.

### DETAILED DESCRIPTION

The accompanying drawings are provided for illustrative purposes only and are not to be construed as limiting the scope of this patent.

To better illustrate the embodiments, certain components in the accompanying drawings may be omitted, enlarged, or reduced in scale, and do not represent the actual dimensions of the product.

It is understandable that for a person skilled in the art, the omission of certain well-known structures and descriptions thereof in the accompanying drawings may be acceptable.

The technical solutions of the disclosure are further described with reference to the accompanying drawings and embodiments below.

### Embodiment 1

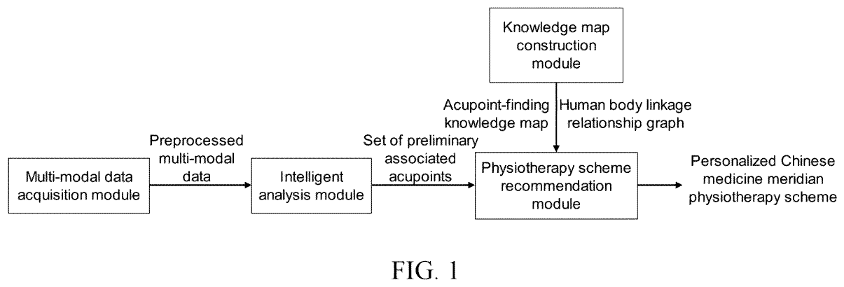

As shown in FIG. 1, this embodiment provides an intelligent recommendation system for a Chinese medicine meridian physiotherapy scheme targeting chronic pain, which includes a multi-modal data acquisition module, a knowledge map construction module, an intelligent analysis module, and a physiotherapy scheme recommendation module.

The multi-modal data acquisition module is sequentially connected to the intelligent analysis module and the physiotherapy scheme recommendation module; and the knowledge map construction module is connected to the physiotherapy scheme recommendation module.

The multi-modal data acquisition module is configured to collect and preprocess multi-modal data from a patient with chronic pain, the multi-modal data at least including medical history information, a pain location, a pain type, and personalized movement examination feedback data.

The knowledge map construction module is configured to construct an acupoint-finding knowledge map and a human body linkage relationship graph on the basis of preset Chinese medicine meridian physiotherapy experience data.

The intelligent analysis module is internally preset with a multi-modal attention network, and the multi-modal attention network is configured to perform feature extraction, feature fusion, and preliminary prediction of the acupoints on preprocessed multi-modal data, and generate a set of preliminary associated acupoints.

The physiotherapy scheme recommendation module is configured to obtain a general physiotherapy scheme on the basis of the acupoint-finding knowledge map, input the general physiotherapy scheme, the set of preliminary associated acupoints, and the human body linkage relationship graph into a preset optimization and calibration network, and ultimately generate a personalized Chinese medicine meridian physiotherapy scheme and complete intelligent recommendation.

During the specific implementation process, this system constructs an acupoint knowledge map on the basis of Chinese medicine meridian physiotherapy experience data, converts a human body linkage relationship into mathematical language understandable by a network, forming a human body linkage inference rule. The system then utilizes the established knowledge map and the human body linkage inference rule, in conjunction with patient movement examinations, chief complaint medical record information, and a set of associated acupoints calculated by the model, to ultimately formulate a personalized physiotherapy scheme.

The system centers on the fusion and analysis of multi-modal data. By multi-modal feature extraction and fusion, the system precisely analyzes pain characteristics of the patient, and optimizes and calibrates a personalized inquiry scheme by integrating the knowledge map and the human body linkage relationship graph. The detailed steps are as follows.

The multi-modal data acquisition module is used to collect multi-modal data from a patient, including chief complaints, movement examinations, pain intensity, and pain coronal plane information.

1) Chief complaint information: the chief complaints of the patient are inputted into an electronic medical record, including a primary pain area, an accompanying pain area, pain intensity, pain duration, and past pain history, so that the electronic medical record of the patient is initially established.

2) Movement examination: pain and functional impairments are assessed by asking the patient to perform specific movements, and VAS is used to evaluate the pain intensity to quantify the pain intensity.

The VAS represents the pain intensity on a straight line (typically 10 centimeters or 100 millimeters in length), with one end representing “no pain” and the other end representing “the most intense pain”. The patient marks the corresponding position on the line on the basis of perceived pain intensity. Medical personnel then assess the pain intensity on the basis of the marked position. The VAS score results can be continuous numerical values, allowing for a more detailed reflection of pain changes. The VAS typically divides pain into 10 levels, with 0 points indicating no pain, 1-3 points indicating mild pain, 4-6 points indicating moderate pain, and 7-10 points indicating severe pain. This score is used to quantify pain changes before and after treatment, providing an intuitive assessment of the therapeutic effects of an intelligent Chinese medicine physiotherapy instrument and verifying the accuracy of the system-generated scheme.

On the basis of the clinical experience and knowledge of professional physicians, the above information is converted into a model input, and the following steps are included.

Preprocessing: the multi-modal data including the chief complaints, movement examination results, pain intensity, and pain distribution of the patient is digitally encoded. For instance, different pain locations can be coded as different numbers, and the pain intensity can be quantified according to the VAS score. After coding, the data is inputted into the multi-modal attention network within the intelligent analysis module.

Feature extraction: the multi-modal attention network is used to extract key features from the coded information. For example, outliers in the range of joint motion are extracted from movement examinations, and primary pain areas are extracted from the pain distribution.

Feature weighting: a multi-head self-attention mechanism is used to adaptively increase the focus of the network on key information. For example, in the case of joint pain, pay more attention to the joint-related examination results.

Feature fusion: on the basis of the mutual attention mechanism, coded vectors of different pain symptom information are fused.

Preliminary prediction: projection and dimensionality reduction of the fused feature is performed by the multi-layer perceptron, and a unique set of preliminary associated acupoints that is in line with the current condition of the patient is outputted.

The knowledge map construction module is used to construct a knowledge map on the basis of the inquiry experience of professional physicians, and link the pain locations, pain nature and movement examination results to possible physiotherapy schemes. For example, if a patient complains of waist pain and has pain distribution in the coronal plane, the knowledge map can search for a general physiotherapy scheme suitable for waist pain patients with coronal plane pain distribution. Moreover, in this module, a human body linkage relationship graph is obtained from a Chinese medicine expert experience summary.

A personalized physiotherapy scheme is generated by combining the unique set of preliminary associated acupoints (acupoints related to the current condition of the patient in this treatment) outputted by the intelligent analysis module with the general physiotherapy scheme (without personalized differences) searched from the acupoint-finding knowledge map and the human body linkage relationship graph. In this embodiment, a graph attention network is used to optimize and calibrate acupoint features and finally output the personalized physiotherapy scheme, ensuring that the physiotherapy scheme aligns with the specific physical condition of the patient.

This system provides a more precise and personalized physiotherapy scheme, achieving personalized inquiries while improving inquiry efficiency and reducing the workload of physicians. Moreover, it does not require patients to wear fixed devices for data collection during the movement examination stage. The system is simple in operation and easy in promotion, enabling non-professionals to conduct self-pain inquiries and rehabilitation management.

### Embodiment 2

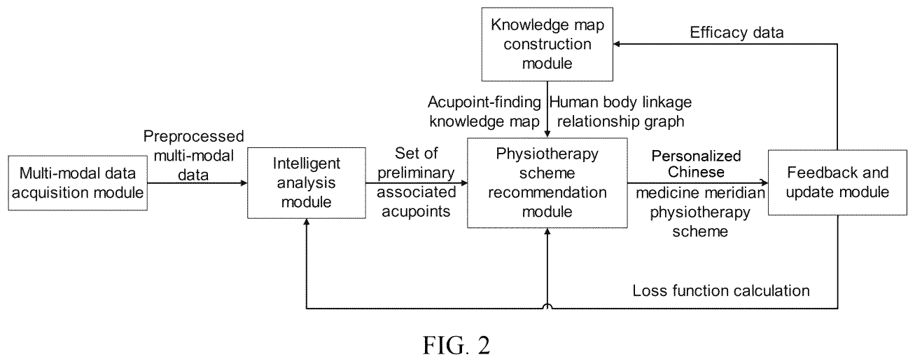

As shown in FIG. 2, this embodiment provides an intelligent recommendation system for a Chinese medicine meridian physiotherapy scheme targeting chronic pain, which includes a multi-modal data acquisition module, a knowledge map construction module, an intelligent analysis module, a physiotherapy scheme recommendation module, and a feedback and update module.

The multi-modal data acquisition module is sequentially connected to the intelligent analysis module and the physiotherapy scheme recommendation module; and the knowledge map construction module is connected to the physiotherapy scheme recommendation module.

The multi-modal data acquisition module is configured to collect and preprocess multi-modal data from a patient with chronic pain, the multi-modal data at least including medical history information, a pain location, a pain type, and personalized movement examination feedback data.

The knowledge map construction module is configured to construct an acupoint-finding knowledge map and a human body linkage relationship graph on the basis of preset Chinese medicine meridian physiotherapy experience data.

The intelligent analysis module is internally preset with a multi-modal attention network, and the multi-modal attention network is configured to perform feature extraction, feature fusion, and preliminary prediction of the acupoints on preprocessed multi-modal data, and generate a set of preliminary associated acupoints.

The physiotherapy scheme recommendation module is configured to obtain a general physiotherapy scheme on the basis of the acupoint-finding knowledge map, input the general physiotherapy scheme, the set of preliminary associated acupoints, and the human body linkage relationship graph into a preset optimization and calibration network, and ultimately generate a personalized Chinese medicine meridian physiotherapy scheme and complete intelligent recommendation.

The feedback and update module is configured to update the acupoint-finding knowledge map and perform fine-tuning of the multi-modal attention network and the optimization and calibration network.

In this embodiment, a patient with chronic pain receives treatment according to the personalized Chinese medicine meridian physiotherapy scheme, and feeds back efficacy data to the feedback and update module.

A preset loss function is calculated on the basis of the efficacy data, and the acupoint-finding knowledge map is updated, and the multi-modal attention network and the optimization and calibration network are fine-tuned on the basis of the preset loss function.

The preset loss function is specifically:

$$Loss = min Δ RI ( p )$$

where Loss is a value of the preset loss function; and ΔRI(p) is a rehabilitation index obtained on the basis of the efficacy data.

The multi-modal data is acquired on the basis of chief complaints of the patient, and the medical history information at least includes chief complaint information about a pain area, chief complaint information about an accompanying pain area, chief complaint information about pain intensity, chief complaint information about pain duration, chief complaint information about past medical history of pain, treatment records, and medication usage.

The pain location includes at least one of head, neck, scapular region, chest, back, waist, pelvis and hip region, legs, knees, ankles, feet, elbows, wrists, or fingers.

The pain type includes dull pain or sharp pain, where the dull pain at least includes aching pain, distending pain, and stuffy pain, and the sharp pain at least includes bursting pain, cutting pain, pricking pain, burning pain, and colicky pain.

On the basis of the pain area selected by the patient, the patient completes movement examinations in horizontal plane, coronal plane, and sagittal plane according to a preset movement prompt, each movement is scored by VAS, and a VAS score is used as the personalized movement examination feedback data of the patient.

The preprocessing includes: performing information coding on the multi-modal data, the information coding including mapping and standardizing the multi-modal data according to a predefined data model; and

performing duplicate data removal, missing value filling, format error correction, and outlier processing on the multi-modal data after information coding to complete the preprocessing.

In the knowledge map construction module, the preset Chinese medicine meridian physiotherapy experience data is imported into a graph database to form the acupoint-finding knowledge map.

In the knowledge map construction module, on the basis of the preset Chinese medicine meridian physiotherapy experience data, an adjacency matrix graph including mutual strength relationships among at least 90 diagnostic and therapeutic points is constructed according to strength characteristics associated with human body linkages, and the human body linkage relationship graph is obtained; and in the human body linkage relationship graph, nodes represent different acupoints, and edges represent direct associations among acupoints.

The preset Chinese medicine meridian physiotherapy experience data specifically is medical case data related to Chinese medicine meridian physiotherapy obtained from a plurality of sources. In this embodiment, medical case data related to Chinese medicine meridian physiotherapy is acquired from two sources: a hospital online system and offline interviews with physicians. a Chinese medicine meridian physiotherapy experience data in this embodiment specifically is medical case data associated with Chinese medicine skin acupuncture therapy.

In the intelligent analysis module, a structure of the multi-modal attention network includes an embedding layer, a multi-head self-attention layer, a mapping layer, a mutual attention layer, and a first multi-layer perceptron layer which are sequentially connected.

The embedding layer is configured to perform key feature extraction on inputted preprocessed multi-modal data, and map a discrete variable in the preprocessed multi-modal data to a continuous vector space to obtain a multi-modal embedding vector.

The multi-head self-attention layer is configured to perform adaptive feature weighting of the multi-modal embedding vector.

The mapping layer is configured to project the multi-modal embedding vector after passing through the multi-head self-attention layer into a higher-dimensional vector space to obtain a high-dimensional multi-modal vector.

The mutual attention layer, on the basis of the mutual attention mechanism, is configured to perform feature fusion on the high-dimensional multi-modal vector, capture mutual dependencies among different modalities by calculating similarity weights among the different modalities, and generate a fused feature after weighting the mutual dependencies.

The first multi-layer perceptron layer is configured to perform preliminary probability prediction of the acupoints on the basis of the fused feature, and output the set of preliminary associated acupoints formed by top-ranked acupoints in terms of prediction probability.

In the physiotherapy scheme recommendation module, an optimization and calibration network includes a graph attention layer and a second multi-layer perceptron layer which are sequentially connected.

The general physiotherapy scheme, the set of preliminary associated acupoints, and the human body linkage relationship graph are jointly inputted into the graph attention layer, and the graph attention layer is configured to perform relationship modeling and semantic enhancement on the general physiotherapy scheme and the set of preliminary associated acupoints by using the human body linkage relationship graph, and calculate a deep nonlinear feature of a physiotherapy scheme containing human body linkage relationships.

The second multi-layer perceptron layer is configured to optimize and calibrate the deep nonlinear feature, capture an implicit combination pattern of the acupoints, output a final acupoint prediction result, and save the final acupoint prediction result as the personalized Chinese medicine meridian physiotherapy scheme, to complete the intelligent recommendation.

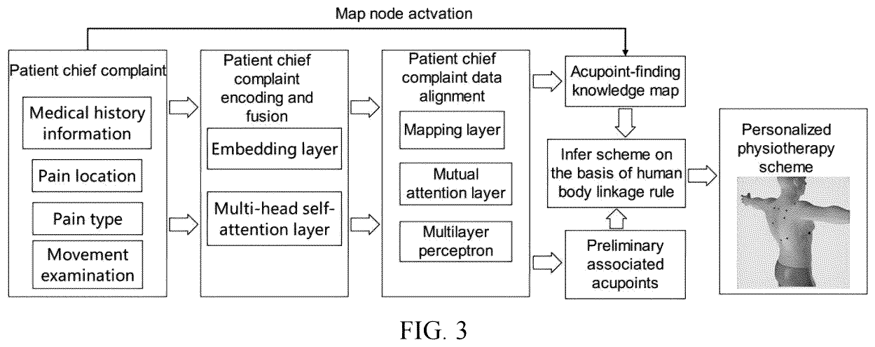

During the specific implementation process, as shown in FIG. 3, this system constructs an acupoint knowledge map on the basis of a Chinese medicine meridian physiotherapy experience data (in this embodiment, the experience of Chinese medicine expert's therapy is selected, which applies weak electrical signal stimulation to specific acupoints on the skin surface to achieve pain relief and regulate the flow of meridian QI and blood, and is applicable to various chronic pain problems, such as neuralgic headache, stomachache, and biliary colic). Moreover, the experience data is converted into a mathematical language understandable by a network, forming a human body linkage inference rule based on a Chinese medicine expert's physiotherapy experience. The system then utilizes the established knowledge map to automatically learn inference relationship of acupoint treatment points. In conjunction with patient movement examinations, chief complaint medical record information, a personalized physiotherapy scheme is ultimately formulated.

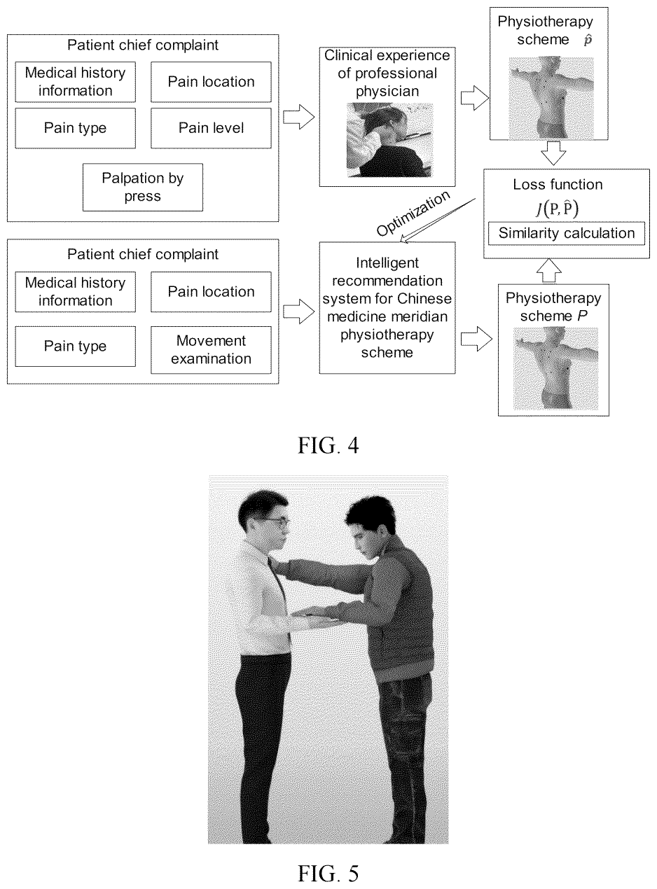

Before formal recommendation, as shown in FIG. 4, it is necessary to pre-train a neural network model within this system. The process is as follows: on the basis of a large number of treatment cases provided by physicians, the model in the system undergoes iterative training. A manually determined physiotherapy scheme and the personalized Chinese medicine meridian physiotherapy scheme generated by the system are utilized. The difference between the two is calculated using a similarity loss function, with cross-entropy loss being employed for this purpose. During the training phase, the system is ensured to learn from a substantial number of cases, so that the recommendation system can achieve physiotherapy scheme recommendations that are essentially consistent with those made by physicians.

The system centers on the fusion and analysis of multi-modal data. By multi-modal feature extraction and data fusion, the system precisely analyzes patient pain characteristics and optimizes and calibrates the personalized inquiry scheme by integrating the knowledge map and the human body linkage relationship graph. The detailed steps are as follows.

First, the multi-modal data acquisition module is used to collect multi-modal data from a patient with chronic pain. The multi-modal data includes medical history information, a pain location, a pain type, and personalized movement examination feedback data.

In this embodiment, the multi-modal data is constructed on the basis of chief complaints of the patient, and the medical history information at least includes chief complaint information about a pain area, chief complaint information about an accompanying pain area, chief complaint information about pain intensity, chief complaint information about pain duration, chief complaint information about past medical history of pain, treatment records, and medication usage. The recommendation system can acquire a comprehensive understanding of a health status of the patient and the individual differences in response to inquiry scheme on the basis of the information, thereby formulating the personalized physiotherapy scheme.

The pain locations refer to the distribution and manifestation of pain across different planes of the body. In this embodiment, the human body is divided into 14 major segments, including head, neck, scapular region, chest, back, waist, pelvis and hip region, legs, knees, ankles, feet, elbows, wrists, and fingers. Understanding the pain locations helps the recommendation system to locate the source of the pain and recommend corresponding physiotherapy schemes on the basis of the source.

The pain type includes dull pain or sharp pain, where the dull pain at least includes aching pain, distending pain, and stuffy pain, and the sharp pain at least includes bursting pain, cutting pain, pricking pain, burning pain, and colicky pain. Understanding the pain type helps the recommendation system to select an inquiry scheme that suits various pain types.

On the basis of the pain area selected by the patient, the system automatically recommends a movement examination scheme in horizontal plane, coronal plane, and sagittal plane. The patient completes movement examinations in horizontal plane, coronal plane, and sagittal plane according to a movement prompt (as shown in FIG. 5), each movement is scored by VAS, and a VAS score is used as the personalized movement examination feedback data of the patient. Through comprehensive analysis of the examination data from the three planar movements, the system can precisely locate the pain area and its impact on specific movements, quantify the degree of movement restrictions of the patient, and use this as a basis to formulate the personalized physiotherapy scheme.

Subsequently, the multi-modal data is preprocessed, including: performing information coding on the multi-modal data, the information coding including mapping and standardizing the multi-modal data according to a predefined data model; and performing duplicate data removal, missing value filling, format error correction, and outlier processing on the multi-modal data after information coding to complete the preprocessing.

The design of the embedding layer primarily aims at transforming discrete pain condition information into low-dimensional continuous vector representations and solving consistency problems in data formats. The embedding layer maps these discrete variables into a continuous vector space to capture the semantic relationships among pain condition information. A structure of the embedding layer can be viewed as a lookup table that maps each discrete category to a fixed-dimensional vector, with a core being a trainable weight matrix. Each row in the weight matrix represents an embedding vector of a category. The specific formula is as follows:

$${e}_{i} = W [ {x}_{i} ] , i ∈ [ 0 , N ]$$

where N=4, and represents the total number of pain condition information (i.e., the total number of modality types in the multi-modal data), xᵢ represents each type of pain condition information, eᵢ represents a vector encoded by the embedding layer, and W represents the weight matrix.

Subsequently, the vector encoded by the embedding layer is inputted into the multi-modal attention network within the intelligent analysis module.

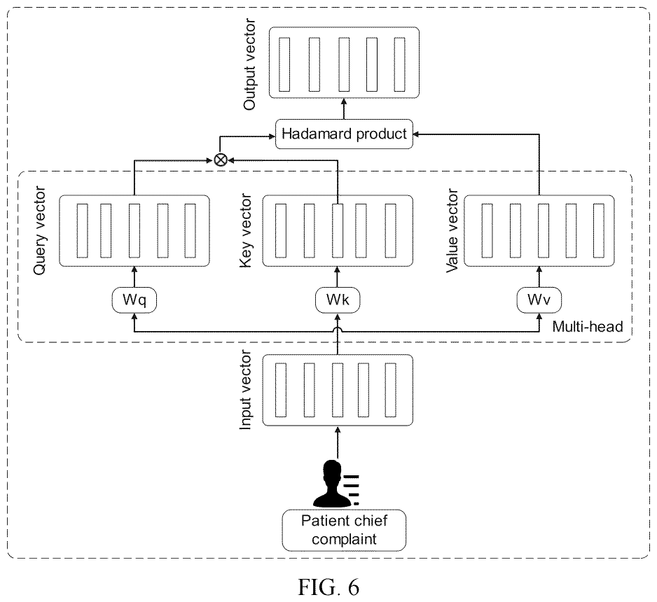

In the previous step, the pain condition information is independently coded into the feature vector by the embedding layer. However, the data itself contains a significant amount of irrelevant components and lacks selective attention to the data of the current modality. Therefore, the multi-modal attention layer utilizes a multi-head self-attention mechanism to adaptively weight its own modal data, enhancing a signal-to-noise ratio of a data feature and increasing attention to feature information significantly relevant to locating pain acupoints. The multi-head design allows each attention mechanism to optimize different feature parts of each modal data, thereby balancing potential biases from a single attention mechanism and enabling the modal data to focus on multi-angle features. The specific layer structure is as shown in FIG. 6.

In the multi-modal attention layer, an encoded vector from each modality is inputted. First, the feature vector is multiplied by weight matrices Wq, Wk, and Wv to obtain a query vector, a key vector, and a value vector, respectively. Their meanings are as follows: the query vector represents the “query” initiated for the current information; the key vector represents a corresponding key in the input feature vector, with each key indicating relevant features thereof; and the value vector represents the actual data or features associated with each input, which the model ultimately uses to generate outputs.

Next, correlation scores between the query vector and the key vector are calculated as weighting coefficients. These scores are subjected to an activation function to obtain attention weight scores, which are subjected to Hadamard product with the value vector, to obtain a pain vector by weighting these weights, thereby enabling the current modal data to focus on those pain input-related features relevant to the current query. This attention mechanism is highly effective in capturing important features, enhancing the ability of the model in understanding complex condition information, thereby providing more reliable data support for accurate diagnosis and personalized physiotherapy scheme design.

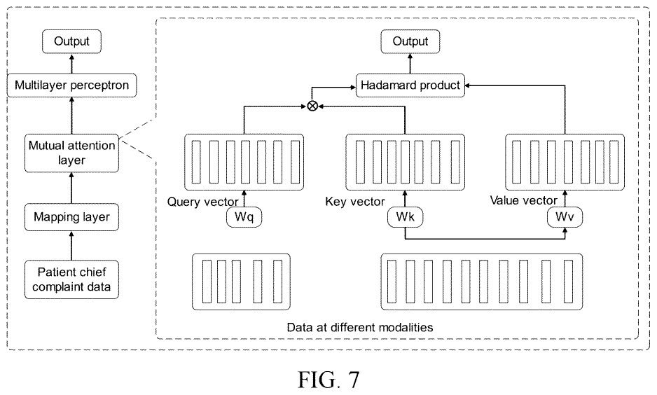

Subsequently, the mutual attention layer is utilized for chief complaint data fusion, mainly to align and fuse multi-modal chief complaint information, solve the difference problem in modality characteristics and distributions, and ensure efficient representation of output features in a unified space, with a structure as shown in FIG. 7. The mutual attention layer is based on the mutual attention mechanism, to perform feature fusion on the high-dimensional multi-modal vector. By calculating similarity weights between different modalities, mutual dependencies among different modalities are captured to generate a weighted fused feature. Specifically, the input multi-modal data includes chief complaint information (such as pain areas, accompanying symptoms, pain intensity, pain duration, and movement examination feedback data) and case information (such as pain medical history, treatment records, and medication usage) of the patient. These input data are first preprocessed and coded to form a plurality of high-dimensional vectors. Then, through the mutual attention mechanism, the model can interact across different modalities, focus on and adjust the influence of each modality, and ultimately generate fused features that comprehensively integrate all aspects of chief complaint and medical record information of the patient.

The condition information features are characterized by discrete data distribution and low feature dimensionality. To enhance the learning ability of the model, in this layer, the condition information features are first projected into a high-dimensional space through the mapping layer. Then, the mutual attention network is used to further fuse multi-modal condition information, establish associations between different data features, and capture their mutual influences and interactions. The core idea of the mutual attention mechanism is to guide feature extraction in one modality by focusing on important information in another modality, playing a crucial role in multi-modal fusion tasks or cross-modal learning tasks. Finally, a multilayer perceptron and a nonlinear activation function are used to further nonlinearly combine and extract high-order features extracted from different modal data. The multilayer perceptron is used to assign probabilities corresponding to physiotherapy schemes for 90 human inquiry points constructed on the basis of the experience of professional physicians, outputting acupoints with probability values exceeding a threshold to obtain a set of preliminary associated acupoints. This set does not incorporate information on the strength of human linkage associations designed on the basis of the experience of professional physicians and requires further adjustment by networks according to expert experience.

Subsequently, the knowledge map construction module is utilized to construct an acupoint-finding knowledge map on the basis of the inquiry experience of professional physicians, linking pain areas, pain properties and movement examination results to possible physiotherapy schemes. For example, if a patient complains of waist pain with pain distribution in the coronal plane, the knowledge map can point to a specific basic physiotherapy scheme for the waist.

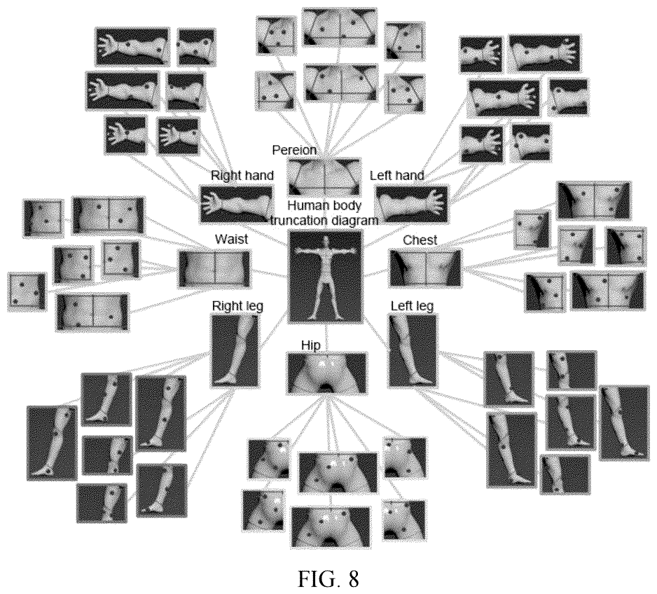

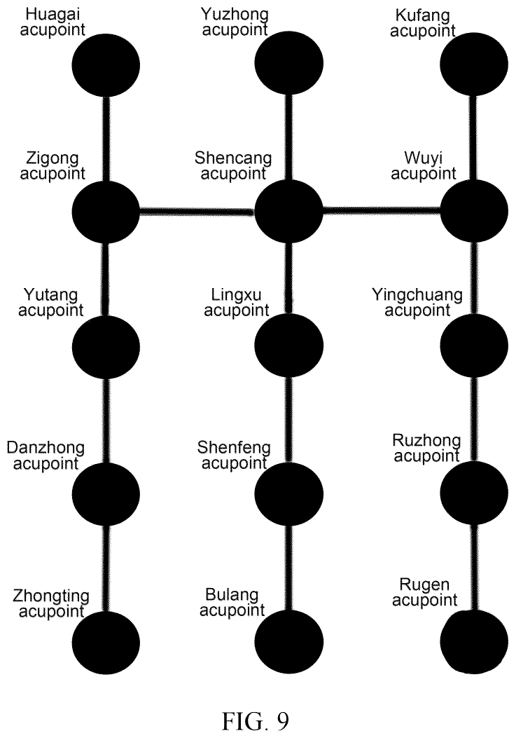

Specifically, in the knowledge map construction module, the preset Chinese medicine meridian therapy experience data is imported into a map database to form the acupoint-finding knowledge map containing nodes and relationships related to pain, intensity, area, physiotherapy schemes, etc., as shown in FIG. 8. On the basis of this map, a basic physiotherapy scheme related to the current condition without personalized differences can be found. Additionally, in the knowledge map construction module, an adjacency matrix graph containing the mutual strength relationships among at least 90 diagnostic and therapeutic points is also constructed on the basis of human linkage inference rule of a Chinese medicine expert's therapy and the strength characteristics associated with human body linkages, as shown in FIG. 9. Nodes in the map represent different acupoints, and edges represent direct associations between acupoints. Following the therapy acupoint constraints (each segment has 2-3 diagnostic and therapeutic points on the horizontal plane, coronal plane, and sagittal plane; distal points are the wrist and ankle, totaling 12 diagnostic and therapeutic points), the logical relationships for acupoint seeking in the therapy are obtained. This module transforms the therapy experience into mathematical language understandable by the network, forming the knowledge rule.

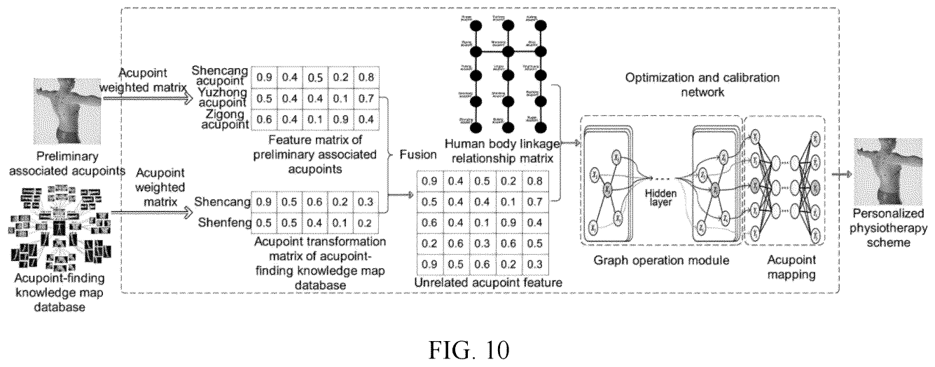

Finally, the physiotherapy scheme recommendation module utilizes the set of associated acupoints inferred by the intelligent analysis module, combined with the basic physiotherapy scheme searched from the knowledge map constructed on the basis of the clinical experience of professional physicians and the human linkage relationship graph, to calculate and generate the personalized physiotherapy scheme. In this embodiment, the graph attention network is used to optimize and calibrate the preliminary physiotherapy scheme to ensure it aligns with the specific physical condition of the patient, as shown in FIG. 10. The associated acupoint feature points and the general physiotherapy scheme searched from the knowledge map are jointly inputted into the graph attention layer, which is used to model relationships and enhance semantics between the general physiotherapy scheme and preliminary associated acupoints using the human linkage relationship graph, thereby calculating a deep nonlinear feature of the physiotherapy scheme containing human linkage relationships. According to the three elements of the self-attention mechanism, graph attention also includes three elements Q, K, and V, where Q is a feature vector of a central node, K is a feature vector of all neighboring nodes, and V is a new feature vector of the central node after aggregation operation.

The role of optimization and calibration is to calibrate and optimize inference results by combining the practical experience of physicians when the final physiotherapy scheme is generated. In the physiotherapy scheme recommendation module, the graph attention layer and the multilayer perceptron work together to optimize and calibrate treatment acupoints. First, the graph information and graph attention layer are used to adjust the preliminary physiotherapy scheme. By introducing graph information and the relationships and feature information between acupoints captured by the graph attention network, the model can focus more on important neighboring nodes, thereby improving the ability of the model to provide personalized physiotherapy schemes. Additionally, the model aggregates feature information from neighboring nodes to obtain feature information for the current node, which makes feature transmission more efficient, thereby better capturing changes in relationships and feature information between nodes, and improving the generalization ability of the model. Next, after passing through the multilayer perceptron, deep nonlinear feature extraction is performed on the output of the graph attention layer to further capture a hidden pattern in acupoint combinations and optimize treatment decisions. Through filtering invalid features, multilayer mapping, and feature calibration, the multilayer perceptron outputs the final treatment acupoint set. Overall, the graph attention layer is responsible for “relationship modeling and semantic enhancement”, and the multilayer perceptron is responsible for “feature optimization and personalized decision-making”. Their combination ensures that the scheme is both scientifically accurate and meets personalized needs.

Through a series of network architectures, the network can generate a personalized physiotherapy scheme based on chief complaint information of the patient and Chinese medicine experience of professional physicians. This scheme not only integrates the relationships between human meridian structures and inquiry points established based on the experience of physicians, but also uses personalized schemes provided by inquiry systems based on professional physicians as supervision to ensure that the network can provide a physiotherapy scheme that meets needs of patients.

To further optimize the scheme, a feedback and update mechanism is designed in this embodiment. After the patient receives treatment, the system collects efficacy data (such as degree of pain relief, improvement in motion limitation, etc.) to dynamically update a Chinese medicine expert's meridian acupoint experience knowledge map, continuously improving the accuracy of the acupoint-finding network model and the quality of the personalized physiotherapy scheme.

Specifically, the learning process of the model for Chinese medicine expert's skin acupuncture inquiry recommendation technology mainly consists of two parts: forward inference and feedback update.

The forward inference process for a personalized Chinese medicine expert's skin acupuncture inquiry scheme is gradually generated by the model on the basis of multi-modal information input. First, patient chief complaint data (such as medical record information, pain locations, pain types, and movement examination data) is inputted into the network. Next, the embedding layer is used to encode the input information, transforming discrete pain condition information into low-dimensional continuous vector representations, and the multi-modal attention network is used to fuse various encoded vectors of different pain condition information to capture semantic relationships among pain condition information. Then, on the basis of the learned pain features, combined with the medical knowledge map of Chinese medicine expert's skin acupuncture, the model can generate a basic physiotherapy scheme and use graph information and the graph attention network to adjust the basic physiotherapy scheme, thereby improving the ability of the model to provide personalized physiotherapy schemes. Finally, the network can generate a personalized physiotherapy scheme that integrates patient chief complaint information and Chinese medicine experience of professional physicians, completing the forward inference process of the model. Moreover, the model learns the fusion relationships of different modality features and deep associations between conditions and acupoints from multi-modal input data and Chinese medicine expert's experience acupoint knowledge map.

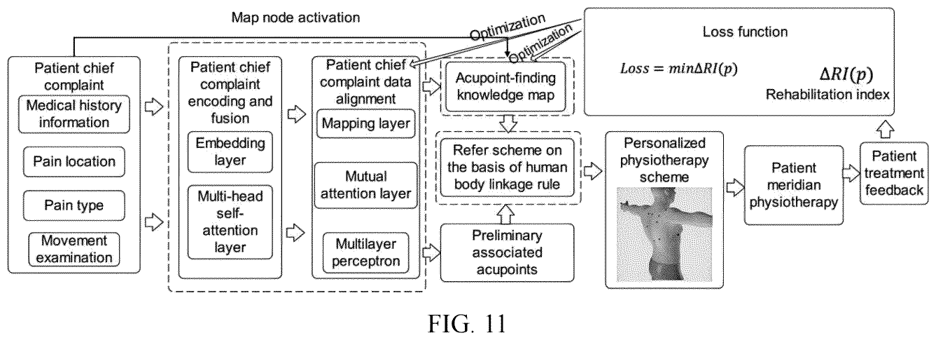

Feedback update is the core mechanism for the model to learn real inquiry techniques and gradually improve (as shown in FIG. 11). After the model generates an inquiry scheme, the patient receives treatment at the recommended acupoints and records treatment effects through efficacy evaluation methods. These efficacy data, such as degree of pain relief and post-treatment recovery, are used as feedback information to be inputted into the system, for further model optimization. After feedback data are inputted into the model, the calibration results are analyzed in-depth by the graph attention mechanism, extracting more accurate feature information from Chinese medicine expert's experience acupoint knowledge map. Moreover, the model further adjusts parameters in each module to enable more accurate alignment of associations between conditions and acupoints of the patient. Through this dynamic update process, the system gradually learns how to better handle complex or special cases, improving its ability to handle diverse patient needs. Additionally, feedback information is also used to update the experience knowledge map. The system re-evaluates the applicability of acupoints on the basis of actual treatment effect feedback, reinforcing records of efficient acupoints, while marking and reducing the recommendation priority of less effective points. Through dynamic adjustment of the knowledge map, the model can gradually form more personalized and targeted inquiry paths.

Through forward inference and feedback update, the system can combine initial condition information and feedback data of the patient to generate an optimized physiotherapy scheme based on personalized condition information of the patient and Chinese medicine experience, while continuously improving the knowledge map representation, and enhancing understanding of deep associations between conditions and acupoints. The forward inference and feedback update form a closed-loop system, and with the accumulation of patient samples, the model continuously learns from new cases and real-time treatment data, not only improving the accuracy and personalization level of inquiry schemes but also optimizing its knowledge representation and decision logic through continuous learning, forming a more dynamic and intelligent inquiry recommendation system.

This system provides a more accurate and personalized physiotherapy scheme, achieving personalized inquiries while improving inquiry efficiency and reducing the workload of physicians. Moreover, it does not require patients to wear fixed devices for data collection during the movement examination stage. The system is simple in operation and easy in promotion, enabling non-professionals to conduct self-pain inquiries and rehabilitation management.

### Embodiment 3

This embodiment provides a validation experiment to verify the effectiveness of the recommendation system described in Embodiment 1 or 2.

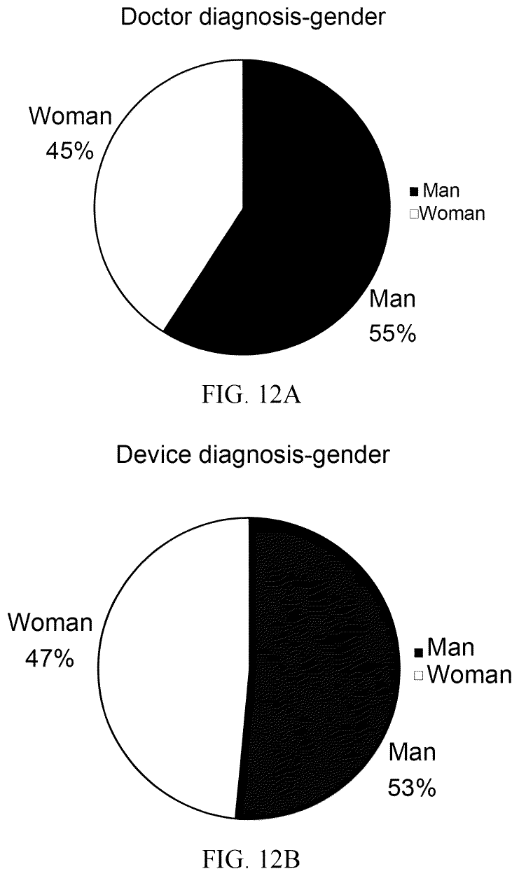

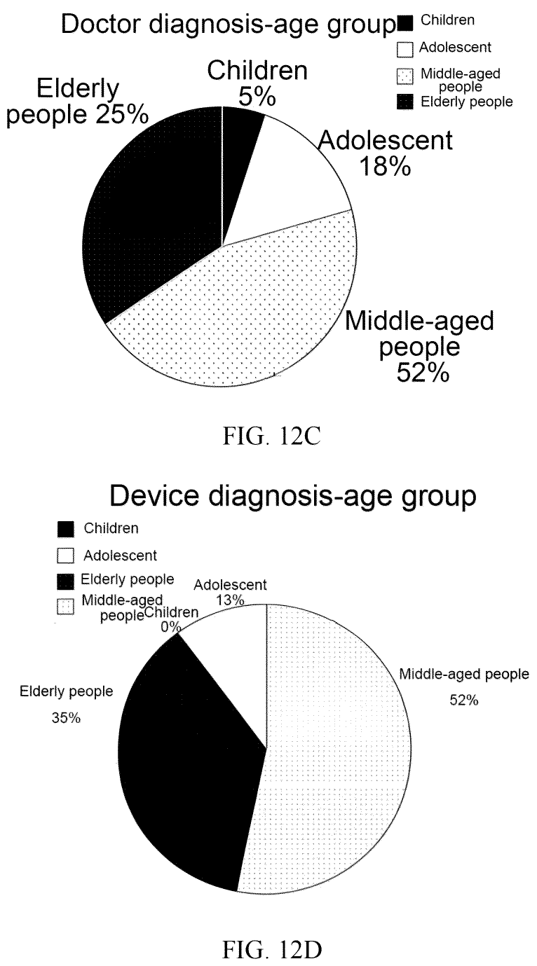

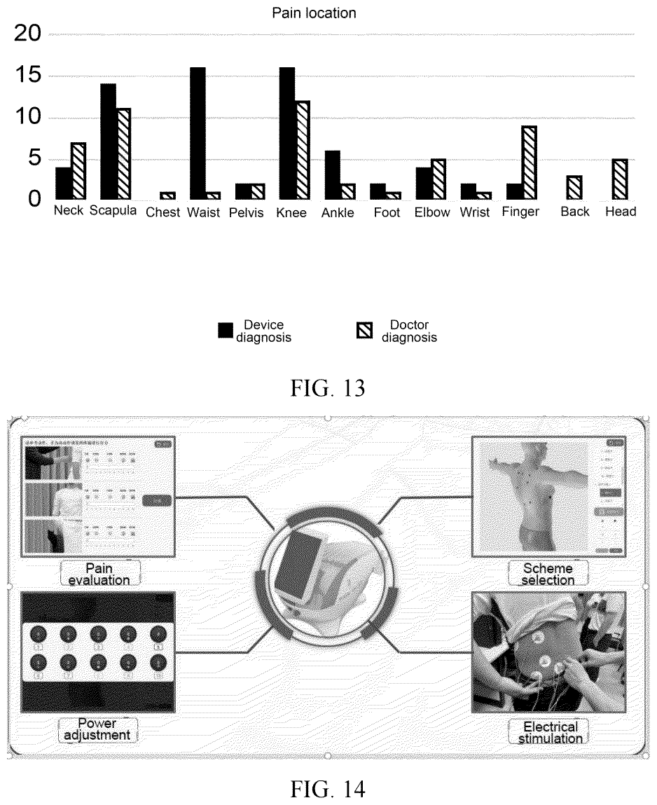

During the specific implementation process, 128 patients with chronic pain are recruited for inquiry in this experiment, with an experimental group including 68 patients who are inquired using the recommendation system provided in Embodiment 1 or 2, and a control group including 60 patients who are personally inquired by professional physicians. Statistical information regarding the gender, age groups, and pain locations of the patients is shown in FIGS. 12A-12D and FIG. 13. It can be seen that there are no significant differences between the experimental group and the control group in terms of gender, age groups, and pain location statistics. The degree of pain relief is calculated on the basis of VAS scores before and after treatment, and the VAS scores are used as the efficacy data to verify the accuracy and effectiveness of the inquiry outcomes.

### Device Introduction:

A core device for this experiment is an intelligent Chinese medicine meridian physiotherapy instrument (as shown in FIG. 14), which is internally arranged with Chinese medicine expert's experience knowledge map and a multi-modal inquiry scheme recommendation network, integrates a chronic pain management inquiry system, and supports multi-point electrical stimulation therapy. The device is designed in a non-invasive manner, utilizing electrode patches to replace traditional acupuncture stimulation. It can precisely stimulate 10 independent treatment points, with 40 levels of current adjustment being provided for each point and covering various therapeutic modes including dilatational waves, discontinuous waves and continuous waves. During treatment, the device can dynamically monitor the physiological responses of the patients and generate feedback data, to support subsequent optimization of inquiry schemes. The device is operated by professional physicians, and in the following comparative experiment, the same treatment device is used. The difference lies in that a built-in chronic pain management inquiry system is used in the experimental group for physiotherapy scheme recommendation, while professional physicians provide physiotherapy schemes on the basis of their own experience in the control group.

### Operation Procedure:

Patients first complete their personal information and medical inquiry records, including pain locations, pain types, duration, and VAS scores. The VAS is an internationally recognized tool for quantifying pain intensity. Patients subjectively express their pain intensity by marking on a straight line ranging from “no pain” (0 points) to “severe pain” (10 points). 0 points indicate no pain, 1-3 points represent mild pain, 4-6 points indicate moderate pain, and 7-10 points represent severe pain. This score is used to quantify changes in the pain of patients before and after treatment, providing a straightforward assessment of the therapeutic effectiveness of the intelligent Chinese medicine physiotherapy instrument and verifying the accuracy of the scheme generated by the system.

Patients in the experimental group undergo movement examinations and pain provocation assessments. The intelligent Chinese medicine meridian physiotherapy instrument precisely locates treatment points on the basis of the data inputted by the patients and the inquiry schemes generated by the system, and then implements electrical stimulation therapy via attached electrode patches. During treatment, the instrument dynamically adjusts therapeutic parameters in real-time through multi-level regulation and multi-channel control to ensure personalized inquiry outcomes. After treatment, patients complete inquiry records again for evaluating pain relief, and treatment efficacy is analyzed in conjunction with data recorded by the instrument. In contrast, patients in the control group are personally diagnosed and treated by professional physicians on the basis of inquiry results. After treatment, patients complete inquiry records again for assessing pain relief.

Statistical analysis of post-physiotherapy data for experimental group and control group:

The results show that among the patients diagnosed and treated using the intelligent Chinese medicine meridian physiotherapy instrument, the proportion of patients with degree of pain relief at 4-10 points reaches 85.29%, compared to 80% of patients personally diagnosed by professional physicians. The proportion of patients inquired using the device with the degree of pain relief at 7-10 reaches 29.41%, compared to 18.33% of patients inquired by the professional physicians. These findings indicate that the inquiry system designed in this patent outperforms the inquiry by traditional physicians. Additionally, the vast majority of patients state significant reductions in pain intensity, expanded range of motion, and a comfortable, well-tolerated treatment experience.

The data statistics results are as shown in Table 1:

Table 1 presents comparison of the degree of relief after
diagnosis with Chinese medicine physiotherapy between
an experimental group and a control group as follows:

| Degree of | Experimental group | | Control group | |
|---|---|---|---|---|
| relief | (68 patients) | Proportion | (60 patients) | Proportion |
| 1-3 | 10 | 14.71% | 12 | 20.00% |
| 4-6 | 38 | 55.88% | 37 | 61.67% |
| 7-10 | 20 | 29.41% | 11 | 18.33% |

### Experimental Results and Discussion:

The experimental results (Table 1) show that the inquiry schemes generated by the intelligent Chinese medicine meridian physiotherapy instrument based on the recommendation system in Embodiment 1 or 2 slightly outperform those personally formulated by professional physicians in terms of therapeutic efficacy; and both the degree of pain relief and functional recovery in patients meet expectations. The verification results prove that the recommendation system in Embodiment 1 or 2 can effectively replace traditional physician inquiries, possesses broad potential for widespread application, and offers safe and efficient physiotherapy schemes for patients with chronic pain while significantly reducing the workload of physicians.

The same or similar labels correspond to the same or similar components.

The terms for describing the positional relationship in the accompanying drawings are provided for illustrative purposes only and are not to be construed as limiting the scope of this patent.

Obviously, the above-described embodiments of the disclosure are merely used to clearly describe the disclosure, rather than limiting the implementation methods of the disclosure. For a person skilled in the art, various other forms of changes or modifications can be made on the basis of the above description. It is neither necessary nor feasible to exhaustively list all possible implementation methods here. Any modifications, equivalents and improvements made within the spirit and principle of the disclosure are included in the scope of the claims of the disclosure.

1. An intelligent recommendation system for a Chinese medicine meridian physiotherapy scheme targeting chronic pain, comprising a multi-modal data acquisition module, a knowledge map construction module, an intelligent analysis module, and a physiotherapy scheme recommendation module, wherein
the multi-modal data acquisition module is sequentially connected to the intelligent analysis module and the physiotherapy scheme recommendation module; and the knowledge map construction module is connected to the physiotherapy scheme recommendation module;
the multi-modal data acquisition module is configured to collect and preprocess multi-modal data from a patient with chronic pain, the multi-modal data at least comprising medical history information, a pain location, a pain type, and personalized movement examination feedback data;
the knowledge map construction module is configured to construct an acupoint-finding knowledge map and a human body linkage relationship graph on the basis of preset Chinese medicine meridian physiotherapy experience data; and in the knowledge map construction module, the preset Chinese medicine meridian physiotherapy experience data is imported into a graph database to form the acupoint-finding knowledge map;
in the knowledge map construction module, on the basis of the preset Chinese medicine meridian physiotherapy experience data, an adjacency matrix graph comprising mutual strength relationships among at least 90 diagnostic and therapeutic points is constructed according to strength characteristics associated with human body linkages, and the human body linkage relationship graph is obtained; and in the human body linkage relationship graph, nodes represent different acupoints, and edges represent direct associations among acupoints;
the intelligent analysis module is internally preset with a multi-modal attention network, and the multi-modal attention network is configured to perform feature extraction, feature fusion, and preliminary prediction of the acupoints on preprocessed multi-modal data, and generate a set of preliminary associated acupoints;
in the intelligent analysis module, a structure of the multi-modal attention network comprises an embedding layer, a multi-head self-attention layer, a mapping layer, a mutual attention layer, and a first multi-layer perceptron layer which are sequentially connected;
the embedding layer being configured to perform key feature extraction on inputted preprocessed multi-modal data, and map a discrete variable in the preprocessed multi-modal data to a continuous vector space to obtain a multi-modal embedding vector;
the multi-head self-attention layer being configured to perform adaptive feature weighting of the multi-modal embedding vector;
the mapping layer being configured to project the multi-modal embedding vector after passing through the multi-head self-attention layer into a higher-dimensional vector space to obtain a high-dimensional multi-modal vector;
the mutual attention layer, on the basis of the mutual attention mechanism, being configured to perform feature fusion on the high-dimensional multi-modal vector, capture mutual dependencies among different modalities by calculating similarity weights among the different modalities, and generate a fused feature after weighting the mutual dependencies; and
the first multi-layer perceptron layer being configured to perform preliminary probability prediction of the acupoints on the basis of the fused feature, and output the set of preliminary associated acupoints formed by top-ranked acupoints in terms of prediction probability; and
the physiotherapy scheme recommendation module is configured to obtain a general physiotherapy scheme on the basis of the acupoint-finding knowledge map, input the general physiotherapy scheme, the set of preliminary associated acupoints, and the human body linkage relationship graph into a preset optimization and calibration network, and ultimately generate a personalized Chinese medicine meridian physiotherapy scheme and complete intelligent recommendation.

2. The intelligent recommendation system for the Chinese medicine meridian physiotherapy scheme targeting chronic pain according to claim 1, wherein the multi-modal data is acquired on the basis of chief complaints of the patient, and the medical history information at least comprises chief complaint information about a pain area, chief complaint information about an accompanying pain area, chief complaint information about pain intensity, chief complaint information about pain duration, chief complaint information about past medical history of pain, treatment records, and medication usage;
the pain location comprises at least one of head, neck, scapular region, chest, back, waist, pelvis and hip region, legs, knees, ankles, feet, elbows, wrists, or fingers;
the pain type comprises dull pain or sharp pain, wherein the dull pain at least comprises aching pain, distending pain, and stuffy pain, and the sharp pain at least comprises bursting pain, cutting pain, pricking pain, burning pain, and colicky pain; and
on the basis of the pain area selected by the patient, the patient completes movement examinations in horizontal plane, coronal plane, and sagittal plane according to a preset movement prompt, each movement is scored by a visual analog scale (VAS), and a VAS score is used as the personalized movement examination feedback data of the patient.

3. The intelligent recommendation system for the Chinese medicine meridian physiotherapy scheme targeting chronic pain according to claim 1, wherein the preprocessing comprises: performing information coding on the multi-modal data, the information coding comprising mapping and standardizing the multi-modal data according to a predefined data model; and
performing duplicate data removal, missing value filling, format error correction, and outlier processing on the multi-modal data after information coding to complete the preprocessing.

4. The intelligent recommendation system for the Chinese medicine meridian physiotherapy scheme targeting chronic pain according to claim 1, wherein the preset Chinese medicine meridian physiotherapy experience data specifically is medical case data related to Chinese medicine meridian physiotherapy obtained from a plurality of sources.

5. The intelligent recommendation system for the Chinese medicine meridian physiotherapy scheme targeting chronic pain according to claim 1, wherein in the physiotherapy scheme recommendation module, the optimization and calibration network comprises a graph attention layer and a second multi-layer perceptron layer which are sequentially connected;
the general physiotherapy scheme, the set of preliminary associated acupoints, and the human body linkage relationship graph are jointly inputted into the graph attention layer, and the graph attention layer is configured to perform relationship modeling and semantic enhancement on the general physiotherapy scheme and the set of preliminary associated acupoints by using the human body linkage relationship graph, and calculate a deep nonlinear feature of a physiotherapy scheme containing human body linkage relationships; and
the second multi-layer perceptron layer is configured to optimize and calibrate the deep nonlinear feature, capture an implicit combination pattern of the acupoints, output a final acupoint prediction result, and save the final acupoint prediction result as the personalized Chinese medicine meridian physiotherapy scheme, to complete the intelligent recommendation.

6. The intelligent recommendation system for the Chinese medicine meridian physiotherapy scheme targeting chronic pain according to claim 1, further comprising a feedback and update module, wherein the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

7. The intelligent recommendation system for the Chinese medicine meridian physiotherapy scheme targeting chronic pain according to claim 2, further comprising a feedback and update module, wherein the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

8. The intelligent recommendation system for the Chinese medicine meridian physiotherapy scheme targeting chronic pain according to claim 3, further comprising a feedback and update module, wherein the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

9. The intelligent recommendation system for the Chinese medicine meridian physiotherapy scheme targeting chronic pain according to claim 4, further comprising a feedback and update module, wherein the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

10. The intelligent recommendation system for the Chinese medicine meridian physiotherapy scheme targeting chronic pain according to claim 5, further comprising a feedback and update module, wherein the feedback and update module is connected to the knowledge map construction module, the intelligent analysis module, and the physiotherapy scheme recommendation module; and
the feedback and update module is configured to update the acupoint-finding knowledge map and perform fine-tuning of the multi-modal attention network and the optimization and calibration network.

11. The intelligent recommendation system for the Chinese medicine meridian physiotherapy scheme targeting chronic pain according to claim 6, wherein the patient with chronic pain receives treatment according to the personalized Chinese medicine meridian physiotherapy scheme, and feeds back efficacy data to the feedback and update module; and
a preset loss function is calculated on the basis of the efficacy data, and on the basis of the preset loss function, the acupoint-finding knowledge map is updated, and the multi-modal attention network and the optimization and calibration network are fine-tuned.

12. The intelligent recommendation system for the Chinese medicine meridian physiotherapy scheme targeting chronic pain according to claim 11, wherein the preset loss function is specifically:
$$Loss = min Δ RI ( p )$$
where Loss is a value of the preset loss function; and ΔRI(p) is a rehabilitation index obtained on the basis of the efficacy data.
